(19) United States
(12) Patent Application Publication
(10) Pub. No.: US 2014/0295433 A1
(43) Pub. Date: Oct. 2, 2014

(54) Method of Detection Using Nano Carbon Carrier Modified by Ionizing Radiation

(71) Applicant: Institute of Nuclear Energy Research, Atomic Energy Council, Executive Yuan, R.O.C., Taoyuan County (TW)

(72) Inventors: Chun-Ying Chen, Taoyuan County (TW); Kuan-Yin Chen, Taoyuan County (TW); Meng-Jun Fu, Taoyuan County (TW); Chin-Yan Tsai, Taoyuan County (TW); Feng-Huei Lin, Taoyuan County (TW); Chia-Ching Liu, Taoyuan County (TW)

(73) Assignee: Institute of Nuclear Energy Research, Atomic Energy Council, Executive Yuan, R.O.C., Taoyuan County (TW)

(21) Appl. No.: 13/958,735

(22) Filed: Aug. 5, 2013

(30) Foreign Application Priority Data
Apr. 2, 2013 (TW) 102111980

(51) Int. Cl. G01N 33/543; G01N 33/574

(52) U.S. Cl. CPC G01N 33/5434; G01N 33/57407
USPC 435/6.12; 436/501; 435/7.92

(57) ABSTRACT

A detection method for cancer is provided. Magnetic carbon beads are used. The carbon beads are highly specified to a cancer. Surface area of grafted antigen are broadened by grafting functional molecules. Number of antigen is increased on the surface. Thus, the present invention improves sensitivity and accuracy of disease detection and greatly saves cost. The present invention can be applied for sample purification or massive disease detection.

### TECHNICAL FIELD OF THE INVENTION

The present invention relates to a disease detection method; more particularly, relates to using magnetic beads having high specificity to a disease and grafting functional molecule on the magnetic beads to enhance surface for grafting antigen, where efficiency of disease detection using the antigen grafted on the nano beads is obviously improved; and, thus, the present invention can be applied in treatments using disease purification and in clinical regular mass in-vitro quantitative cancer diagnoses.

### DESCRIPTION OF THE RELATED ART

A prior art, enzyme-linked immunosorbent assay (ELISA), is shown in FIG. 9. A layer of an antigen 9 is only coated at bottom of a container 8. Only a limited part of surface of the antigen 9 can be used. Sensitivity of detection cannot be effectively enhanced. Hence, the prior art does not fulfill all users' requests on actual use.

### SUMMARY OF THE INVENTION

The main purpose of the present invention is to use magnetic beads having high specificity to a disease and use grafting functional molecule on the magnetic beads to enhance surface for grafting antigen, where efficiency of disease detection using the antigen grafted on the nano beads is obviously improved; and, thus, the present invention can be applied in treatments using disease purification and in clinical regular mass in-vitro quantitative cancer diagnoses.

To achieve the above purpose, the present invention is a method of detection using a nano carbon carrier modified by ionizing radiation, comprising steps of: (a) obtaining a solution having a plurality of magnetic beads and adding an antigen into the solution to combine the antigen with the magnetic beads, where the magnetic bead is a radioactive nano carrier, comprising a nano bead; a plurality of grafting functional molecules distributed on the nano carbon bead; and a magnetic material distributed on the nano bead; (b) gathering the magnetic beads by using a magnetic field and removing un-reacted part of the antigen and extra part of the solution by an absorber to obtain the solution having the magnetic beads all combined with the antigen; (c) adding an under-testing sample to the solution having the magnetic beads combined with the antigen and processing a specific reaction between a primary antibody of the under-testing sample and the antigen of the magnetic beads to combine the primary antibody with the antigen and gathering the magnetic beads by using a magnetic field to separate and remove uncombined part of the under-testing sample; and (d) adding a secondary antibody to the solution to combine the secondary antibody with the primary antibody and processing a content detection through combining the secondary antibody with a radioactive isotope, an enzyme or a nucleic acid molecule (DNA). Accordingly, a novel method of detection using a nano carbon carrier modified by ionizing radiation is obtained.

### BRIEF DESCRIPTIONS OF THE DRAWINGS

The present invention will be better understood from the following detailed description of the preferred embodiment according to the present invention, taken in conjunction with the accompanying drawings, in which

FIG. 1 is the flow view showing the preferred embodiment according to the present invention;

FIG. 2 is the view showing the step (a);

FIG. 3 is the view showing the step (b);

FIG. 4 is the view showing the step (c);

FIG. 5 is the view showing the step (d);

FIG. 6 is the view showing the step (e);

FIG. 7 is the view showing the step (f);

FIG. 8 is the view showing the step (g); and

FIG. 9 is the view of the prior art.

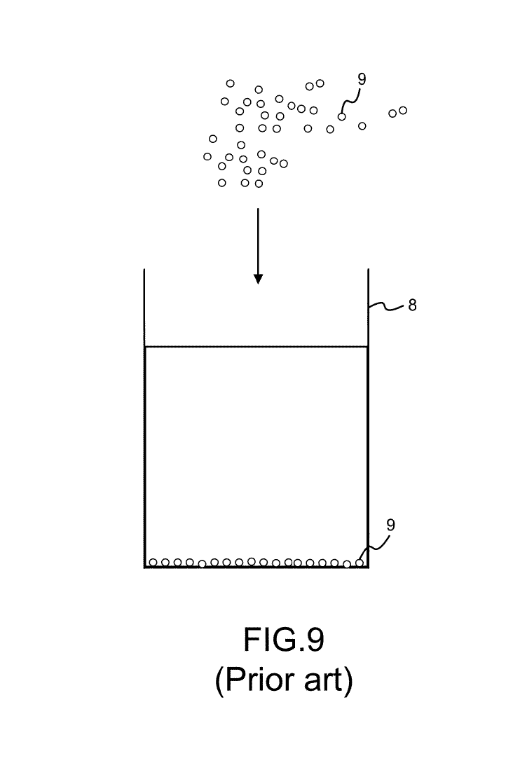

### DESCRIPTION OF THE PREFERRED EMBODIMENT

The following description of the preferred embodiment is provided to understand the features and the structures of the present invention.

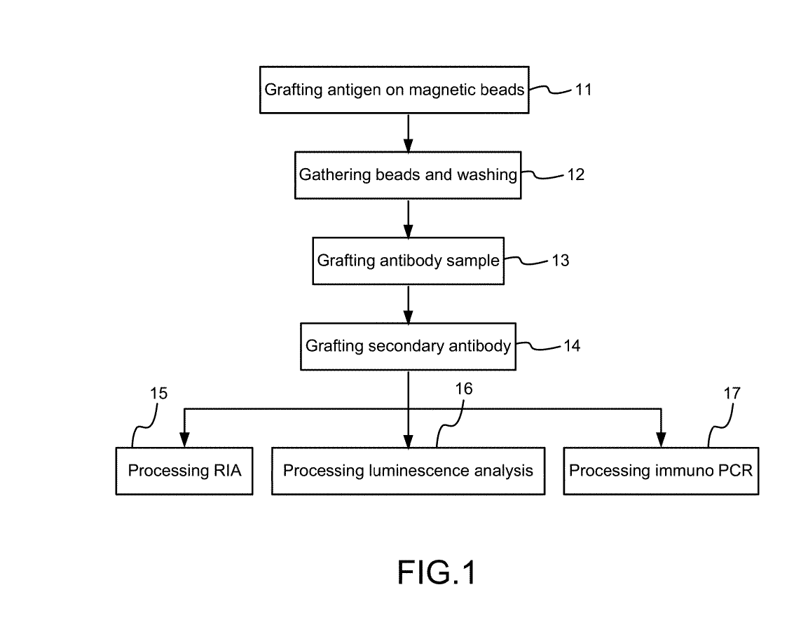

Please refer to FIG. 1 to FIG. 8, which are a flow view showing a preferred embodiment according to the present invention and views showing step (a) to step (g). As shown in the figures, the present invention is a method of detection using a nano carbon carrier modified by ionizing radiation, comprising the following steps:

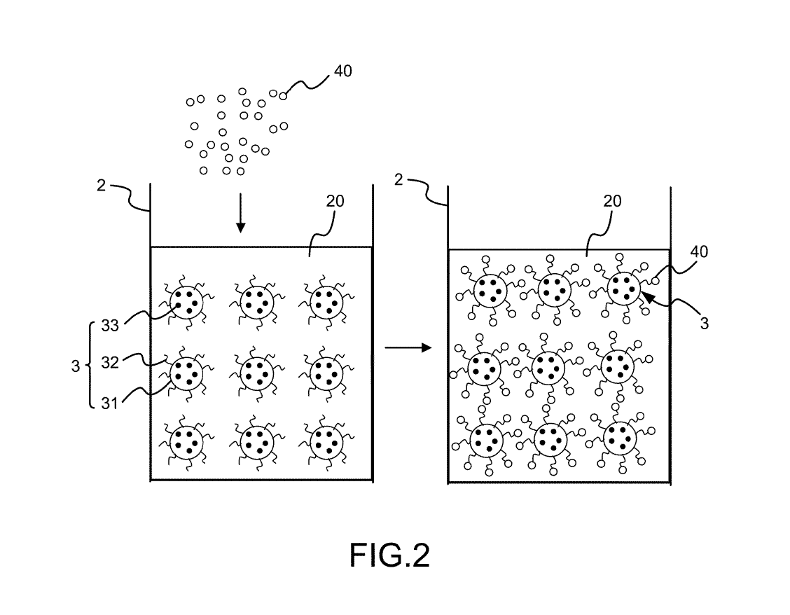

(a) Grafting antigen on magnetic beads 11: In FIG. 2, a plurality of magnetic beads 3 is provided in a solution 20 contained in a container 2 and an antigen 40 is added into the solution 20 to be grafted with the magnetic beads 3, where the magnetic bead is a radioactive nano carrier, comprising a nano bead 31; a plurality of grafting functional molecules 32 distributed on the nano bead 31; and a magnetic material 33 distributed on the nano bead 31.

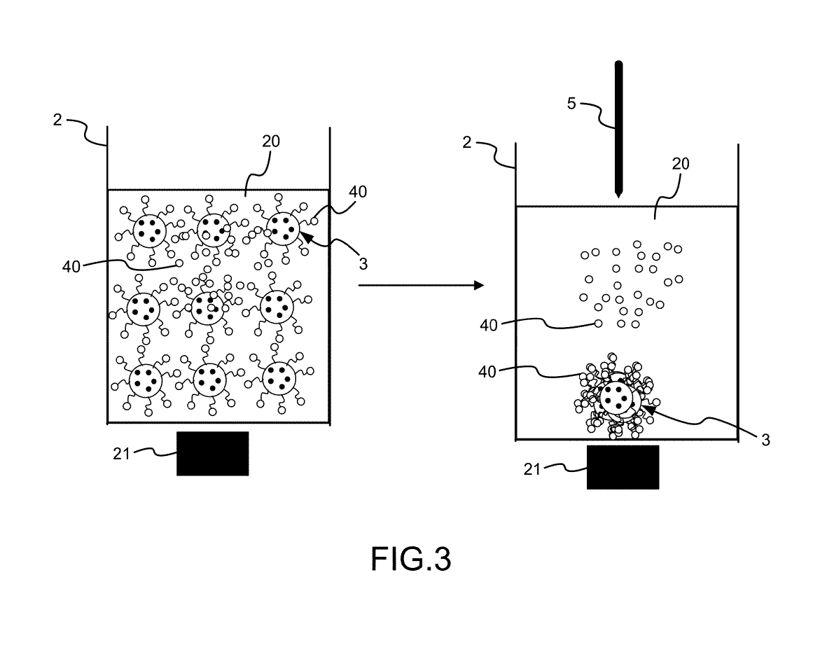

(b) Gathering beads and washing 12: In FIG. 3, a magnet 21 is put under the container 2 for gathering the magnetic beads 3 by using a magnetic field of the magnet 21. Un-reacted part of the antigen 40 and the solution 20 is removed by an absorber (i.e. needle) 5 to form the magnetic beads 3 all grafted with the antigen 40.

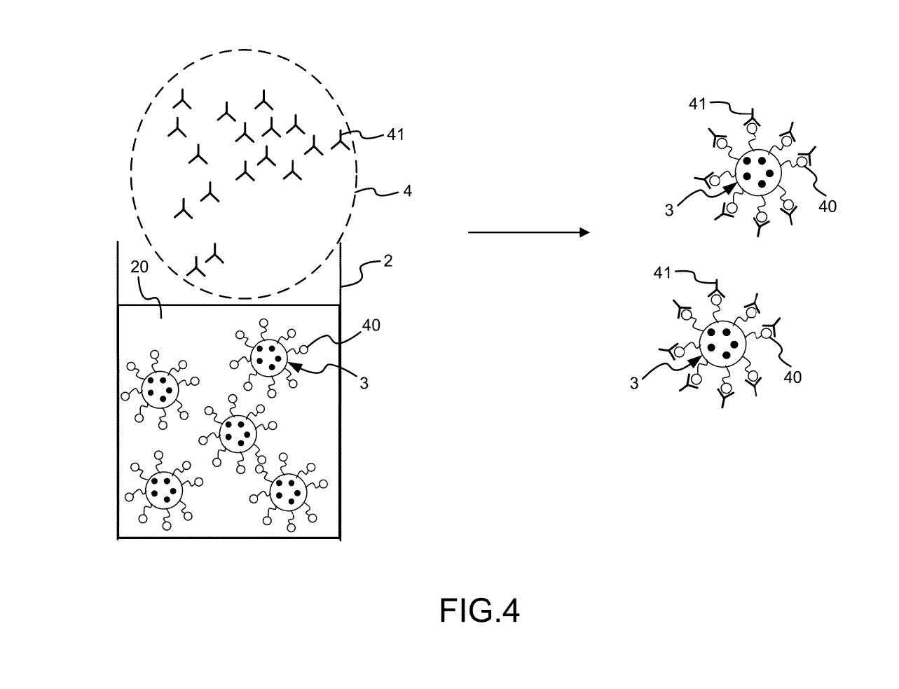

(c) Grafting antibody sample 13: In FIG. 4, an under-testing sample is added into the solution 20 containing the magnetic beads 3 grafted with the antigen 40. A specific reaction is processed between a primary antibody 41 in the under-testing sample 4 and the antigen 40 on the magnetic beads 3 for grafting the primary antibody on the antigen. Then, like what has been done in step (b), the magnetic beads 3 are gathered by using a magnetic field to separate and remove un-grafted part of the under-testing sample 4.

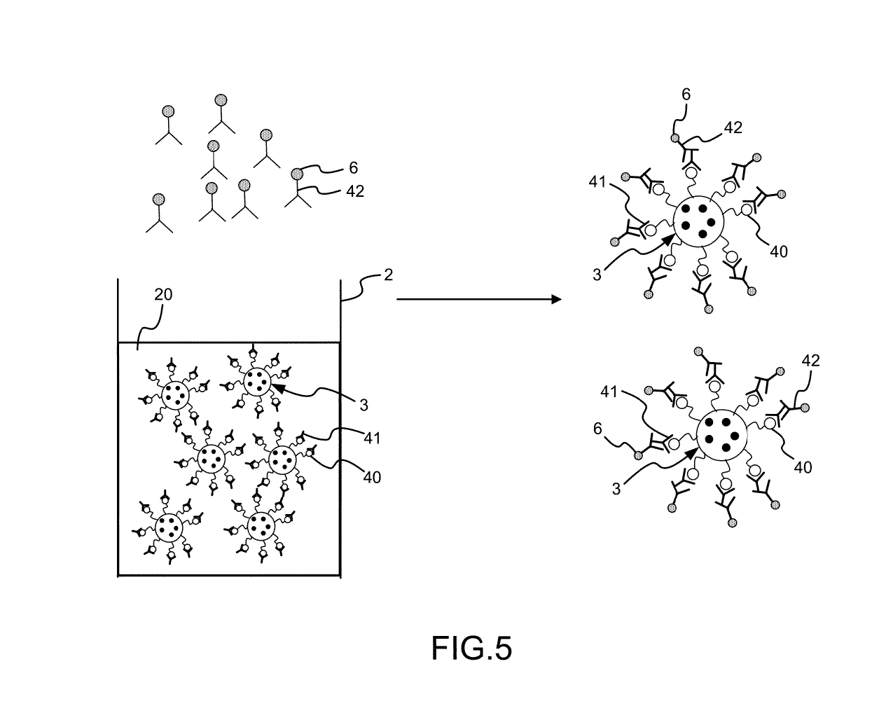

(d) Grafting secondary antibody 14: In FIG. 5, a secondary antibody 42 is added to be grafted with the solution 20 having the magnetic beads 3 for processing a content detection. The content detection is done by grafting the secondary antibody 42 with a signal molecule 6, like a radioactive isotope, an enzyme or a nucleic acid molecule (DNA), as described in step (e), step (f) and step (e).

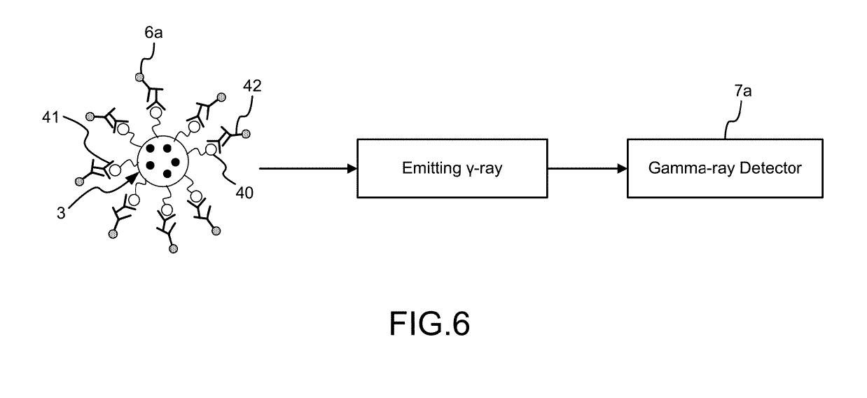

(e) Processing RIA 15: In FIG. 6, radioimmunoassay (RIA) is processed by coupling a radioactive isotope 6a of iodine(I)-125 on the secondary antibody 42 for detecting a strength of gamma ray (γ-ray) radiated from I-125 to obtain a content of the primary antibody 41 in the under-testing sample 4.

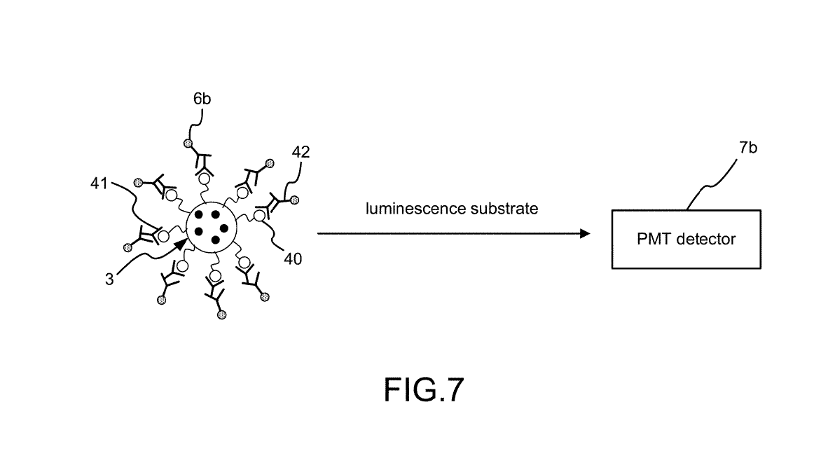

(f) Processing luminescence analysis 16: In FIG. 7, chemiluminescence immunoassay (CLIA) or enzyme-linked immunosorbent assay (ELISA) is processed by coupling a luminescent colorimetric enzyme 6b on the secondary antibody 42 to be reacted with a luminescence substrate for detecting a photon strength of the luminescence substrate to obtain a content of the primary antibody 41 in the under-testing sample 4.

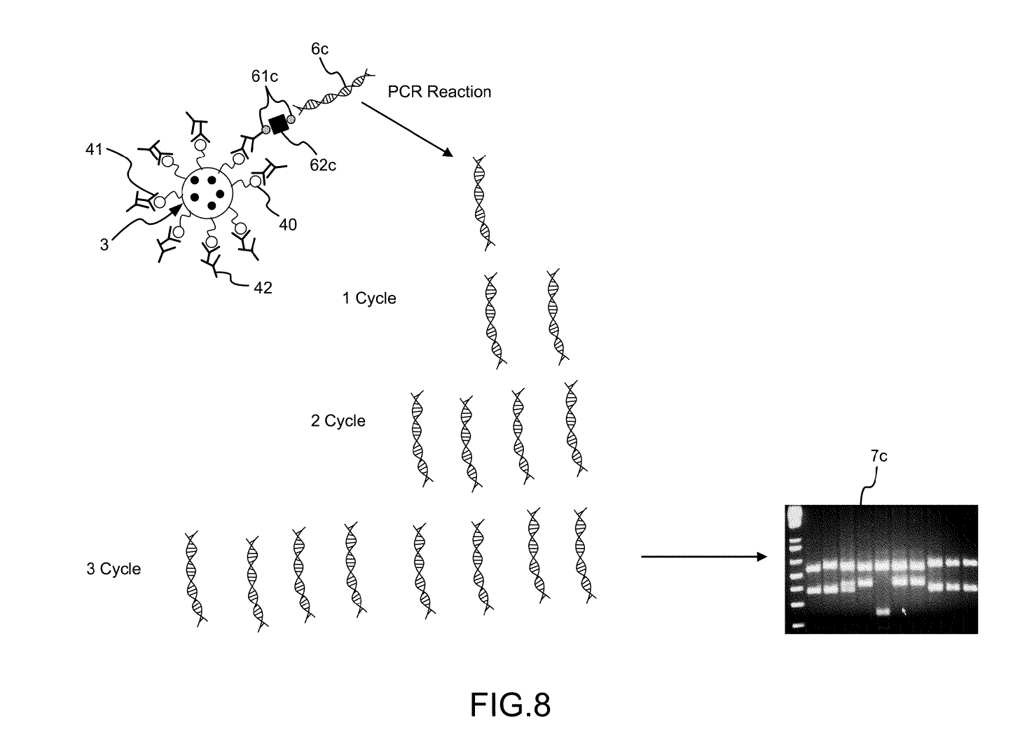

(g) Processing immuno PCR 17: In FIG. 8, immuno polymerase chain reaction (PCR) is processed by combining a biotin 61c on the secondary antibody 42; combining another biotin 61c on a nucleic acid molecule 6c; connecting the nucleic acid molecule 6c with the secondary antibody 42 through streptavidin 62c; processing PCR with a tag enzyme for forming magnified reaction signals; and separating the nucleic acid molecule 6c to obtain a content of the primary antibody 41 in the under-testing sample 4.

The nano bead 31 is a nano carbon bead; the grafting functional molecule 32 has a functional group formed through an acid-alkali treatment and ionizing radiation and the functional group is —COOH, —NH₂, —SH, —OH, —COH or —COO—; and, the magnetic material 33 is a magnet powder made of iron (Fe), cobalt (Co), nickel (Ni) or iron (II, III) oxide, (Fe₃O₄).

Thus, a novel method of detection using a nano carbon carrier modified by ionizing radiation is obtained.

On using the present invention, the magnetic beads 3 are used to combine the antigen 40 on surface of the nano beads 31 by the grafting functional molecule 32. Then, by using the magnet 21 put under the container 2, the magnetic material 33 of the magnetic beads 3 is reacted with the magnetic field of the magnet 21 to be attracted and moved toward the magnet 21 and, then, an absorber 5 is used to absorb extra part of the solution. Thus, un-reacted part of the antigen 40 can be easily removed by washing to form the magnetic beads 3 all combined with the antigen 40. Then, the under-testing sample 4, i.e. serum of a nasopharyngeal cancer patient, is added. By using the magnetic beads 3 having adhesion specificity to the cancer, only the primary antibody in the under-testing sample 4, i.e. anti-EBV IgA, is grafted on the magnetic beads 3. In the same way, a magnetic field is used to gather the magnetic beads 3 and the other part of the serum is separated and removed. Then, the magnetic beads 3 grafted with anti-EBV IgA is grafted with the secondary antibody 42, i.e. anti-Human IgA. Again, a magnetic field is used to gather the magnetic beads 3 for separating and removing the other un-adhered part of the secondary antibody 42. At last, the secondary antibody 42 can be grafted with one of three different signal molecule 6 for processing RIA, a luminescence analysis or immuno PCR.

On processing RIA, the secondary antibody 42 is coupled with a radioactive isotope 6a of I-125. γ-ray will be radiated from I-125. By detecting a γ-ray strength through a γ-ray detector, a content of the primary antibody (anti-EBV IgA) in the under-testing sample 4 is obtained. This detection method has high accuracy and low cost.

On processing luminescence analysis, the secondary antibody 42 is coupled with a luminescent colorimetric enzyme 6b, like horse radish peroxidase (HRP) or alkaline phosphatase (AP). The luminescent colorimetric enzyme 6b is reacted with a luminescence substrate. A photomultiplier tube (PMT) detector 7b is used to detect a photon strength to obtain a content of the primary antibody 41 in the under-testing sample 4. This detection method obtains magnified signals for good accuracy; and, furthermore, can achieve convenience by using an automatic serum immuno analyzer.

On processing immuno PCR, the secondary antibody 42 is grafted with the biotin 61c. At the same time, the nucleic acid molecule 6c is grafted with another biotin 61c. Streptavidin 62c has strong adhesion to biotin and can be grafted with four biotins. Therefore, streptavidin 62c is used to connect the nucleic acid molecule 6c with the secondary antibody 42. Then, PCR is processed with a tag enzyme for obtaining magnified reaction signals. Then, gel electrophoresis 7c is used to separate the nucleic acid molecule 6c for obtaining a content of the primary antibody 41 in the under-testing sample 4. This detection method can magnify reaction signals for 1 billion times after 30 cycles and, thus, is the most sensitive detection method.

Hence, the present invention uses magnetic beads for obtaining high specificity to a disease, where grafting functional molecule is used to enhance surface for grafting antigen. Efficiency of ELISA for the antigen grafted on the nano beads is obviously improved. Consequently, the present invention can be applied in treatments using disease purification and in clinical regular mass in-vitro quantitative cancer diagnoses.

To sum up, the present invention is a method of detection using a nano carbon carrier modified by ionizing radiation, where magnetic beads having high specificity to a disease is used and grafting functional molecule is used to enhance surface for grafting antigen; efficiency of detection method using the antigen grafted on the nano beads is obviously improved; and the present invention can be applied in treatments using disease purification and in clinical regular mass in-vitro quantitative cancer diagnoses.

The preferred embodiment herein disclosed is not intended to unnecessarily limit the scope of the invention. Therefore, simple modifications or variations belonging to the equivalent of the scope of the claims and the instructions disclosed herein for a patent are all within the scope of the present invention.

What is claimed is:

1. A method of detection using a nano carbon carrier modified by ionizing radiation, comprising steps of:
(a) obtaining a solution having a plurality of magnetic beads and adding an antigen into said solution to combine said antigen with said magnetic beads,
wherein said magnetic bead is a radioactive nano carrier, comprising
a nano bead;
a plurality of grafting functional molecules, said grafting functional molecules being distributed on said nano carbon bead; and
a magnetic material, said magnetic material being distributed on said nano bead;
(b) gathering said magnetic beads by using a magnetic field and removing un-reacted part of said antigen and extra part of said solution by an absorber to obtain said solution having said magnetic beads all combined with said antigen;
(c) adding an under-testing sample to said solution having said magnetic beads combined with said antigen and processing a specific reaction between a primary antibody of said under-testing sample and said antigen of said magnetic beads to combine said primary antibody of said under-testing sample with said antigen of said magnetic beads and gathering said magnetic beads by using a magnetic field to separate and remove uncombined part of said under-testing sample; and
(d) adding a secondary antibody to said solution having said magnetic beads combined with said primary antibody to combine said secondary antibody with said primary antibody of said under-testing sample and processing a content detection through combining said secondary antibody with a signal molecule selected from a group consisting of a radioactive isotope, an enzyme and a nucleic acid molecule (DNA).

2. The method according to claim 1,
wherein said nano bead is a nano carbon bead.

3. The method according to claim 1,
wherein said content detection is done through radioimmunoassay (RIA) by combining a radioactive isotope on said secondary antibody and detecting a strength of gamma ray (γ-ray) radiated from said radioactive isotope to obtain a content of said primary antibody in said under-testing sample.

4. The method according to claim 3,
wherein said radioactive isotope is iodine(I)-125.

5. The method according to claim 1,
wherein said content detection is done through a luminescence analysis by combining a luminescent colorimetric enzyme on said secondary antibody to be reacted with a luminescence substrate and detecting a photon strength to obtain a content of said primary antibody in said under-testing sample.

6. The method according to claim 5,
wherein said luminescence analysis is selected from a group consisting of chemiluminescence immunoassay (CLIA) and enzyme-linked immunosorbent assay (ELISA).

7. The method according to claim 1,
wherein said content detection is done through immuno polymerase chain reaction (PCR) by combining a biotin on said secondary antibody; combining another biotin on a nucleic acid molecule; connecting said nucleic acid molecule with said secondary antibody through streptavidin; processing PCR with a tag enzyme to obtain magnified reaction signals; and separating said nucleic acid molecule to obtain a content of said primary antibody in said under-testing sample.

8. The method according to claim 1,
wherein said under-testing sample is a serum of a patient of a cancer.

9. The method according to claim 8,
wherein said cancer is nasopharyngeal cancer.

10. The method according to claim 1,
wherein said primary antibody is anti-EBV IgA.

11. The method according to claim 1,
wherein said secondary antibody is anti-Human IgA.

12. The method according to claim 1,
wherein, in step (d), after combining said secondary antibody with said magnetic beads, a magnetic field is used to gather said magnetic beads to separate uncombined part of said secondary antibody.

13. The method according to claim 5,
wherein said luminescent colorimetric enzyme is selected from a group consisting of horse radish peroxidase (HRP) and alkaline phosphatase (AP).

14. The method according to claim 5,
wherein said luminescence analysis is an automatice serum immue analysis.

15. The method according to claim 5,
wherein said photon strength is obtained through a photomultiplier tube (PMT) detector.

16. The method according to claim 7,
wherein said nucleic acid molecule is separated through gel electrophoresis.

17. The method according to claim 1,
wherein said grafting functional molecule has a functional group formed through an acid-alkali treatment and ionizing radiation.

18. The method according to claim 17,
wherein said functional group is selected from a group consisting of —COOH, —NH₂, —SH, —OH, —COH and —COO—.

19. The method according to claim 1,
wherein said magnetic material is a magnet powder made of a material selected from a group consisting of iron (Fe), cobalt (Co), nickel (Ni) and iron(II, III) oxide, (Fe₃O₄).
